SEQUENCE LISTING (4 sequences)

SEQ ID NO: 1 - DNA, 15 bp, Artificial Sequence
  probe sequence immobilized on nanoparticle
tgatgaattctactg

SEQ ID NO: 2 - DNA, 15 bp, Artificial Sequence
  protecting sequence immobilized on nanoparticle
actcattaagattac

SEQ ID NO: 3 - DNA, 27 bp, Artificial Sequence
  linking sequence between target and probe sequence
gggcggcgacctcattagaattcatca

SEQ ID NO: 4 - DNA, 12 bp, Artificial Sequence
  Sequence complementary to part of linking sequence
aggtcgccgccc

(19) United States
(12) Patent Application Publication
(10) Pub. No.: US 2012/0225457 A1
(43) Pub. Date: Sep. 6, 2012

(54) NANOPARTICLE-NUCLEIC ACID COMPLEX AND METHOD OF LINEARIZING TARGET NUCLEIC ACID

(75) Inventors: June-young LEE, Anyang-si (KR); Jwa-min NAM, Seoul (KR); Su-hyeon KIM, Seoul (KR); Woo-chang LEE, Anyang-si (KR); Jae-young KIM, Seoul (KR); Joo-won RHEE, Yongin-si (KR); Young-kwang LEE, Goyang-si (KR); Dong-kwon LIM, Seongnam-si (KR); Jae-ho HWANG, Seongnam-si (KR)

(73) Assignees: SNU R&DB FOUNDATION, Seoul (KR); SAMSUNG ELECTRONICS CO., LTD, Suwon-si (KR)

(21) Appl. No.: 13/294,596

(22) Filed: Nov. 11, 2011

(30) Foreign Application Priority Data
Mar. 4, 2011 (KR) 10-2011-0019648

(51) Int. Cl. C12P 19/34; B32B 5/16; C07H 21/00; B82Y 99/00

(52) U.S. Cl. 435/91.52; 536/24.3; 428/403; 977/773; 977/810; 977/902

(57) ABSTRACT

A nanoparticle-nucleic acid complex and a method of linearizing a target nucleic acid by using the nanoparticle-nucleic acid complex are disclosed. By using the nanoparticle-nucleic acid complex and the method, nucleotide sequence analysis and mapping of a target nucleic acid may be efficiently performed.

### CROSS-REFERENCE TO RELATED APPLICATIONS

This application claims the benefit of Korean Patent Application No. 10-2011-0019648, filed on Mar. 4, 2011, in the Korean Intellectual Property Office, the disclosure of which is incorporated herein by reference.

### BACKGROUND

1. Field

The present disclosure relates to a nanoparticle-nucleic acid complex and a method of linearizing a target nucleic acid with the nanoparticle-nucleic acid complex.

2. Description of the Related Art

Deoxyribonucleic acid (DNA) is a linear polymer of four kinds of nucleotides distinguished by their base, including adenine (A), cytosine (C), guanine (G), and thymine (T). Nucleotide sequence analysis of DNA by existing chain termination methods takes time and incurs tremendous costs, which have been reduced with the development of new technologies, enabling completion of human genome analysis in a week. Recently, new rapid analysis methods for human genomes have been developed, but they cost of hundreds to thousands of dollars, which is still a high cost. DNA sequencing methods using hydromechanics and pore-based systems, are manufactured by BioNanomatrix, NABsys, and the like, and require linearizing long DNA having a length of several thousand bases (kb) to optically or electrically obtain signals providing sequence information. However, hydraulic motion in DNA is difficult to control, and therefore is a challenging issue for efficient DNA linearization and effective nucleotide sequence analysis.

One-to-one binding of target DNA molecules of interest to surfaces of nanoparticles (metal or non-metal) may ensure more efficient linearization of DNA in an analytic system including microchannels, nanofluidic channels, nanopores, thus preventing DNA overlapping and enabling more effective nucleotide sequence analysis. Optical and physical characteristics of the nanoparticles may be used for other advantages such as DNA orientation recognition, DNA immobilization, and optical signal enhancements.

Research has been actively conducted into optical signal amplification by gold nanoparticles due to the unique surface plasmon resonance phenomena of gold nanoparticles, and thus forming gold nanoparticles having various diameters is currently possible. Gold nanoparticles have high affinity to biomolecules and may form stable electrostatic or covalent bonds with thiol (—SH), alcohol (—OH), and amine (—NH₂) groups. Known to be nearly non-toxic in the body, gold nanoparticles are receiving attention as a research topic, especially in the field of biology. With a DNA nucleotide sequence, such as a thiolated oligonucleotide, attached to the surface of a gold nanoparticle, a complementary sequence, bound to another gold nanoparticle, may then be hybridized with the DNA nucleotide sequence, making the two gold nanoparticles get closer, which leads to a change in color of the reaction solution from pink to violet. Reportedly, based on this phenomenon, the nucleotide sequence linking the two adjacent nanoparticles may be detected. Accurate, efficient linearization of macro DNA molecules requires accurate binding of one oligonucleotide to each nanoparticle with a high yield, which needs to involve neither complicated separation nor small-scale methods; rather, it should be simple separation and scalable methods for mass production. About 100-200 oligonucleotides with thiol groups are made to bind to gold nanoparticles having a diameter of 15-20 nm, so binding of only one oligonucleotide to a surface of each metal nanoparticle having a particular size happens rarely. DNA hybridization is generally induced at a high salt concentration (0.1M˜0.3M NaCl). This requires treatment of an oligonucleotide-free surface for stabilization. Recently, research into attaching a polynucleotide with one thiol group to a surface of one metal nanoparticle has been actively performed. According to related existing technologies, a thiolated oligonucleotide is added in a relatively small molar ratio with respect to gold nanoparticles to bind to a relatively stable nanoparticle surface with a phospine-ligand substituent. At least four of the gold nanoparticles bound with the thiolated oligonucleotide, but not bound with polynucleotide, are separated using agarose-gel electrophoresis to isolate bands with only one DNA, which is then reacted with a nanoparticle bound with a complementary nucleotide sequence, followed by identifying binding of one DNA to form a nanodimer. This technology, however, is very low in efficiency and involves complicated separation processes.

DNA molecules have a random coil polymer structure in solution. Thus, to optically identify the presence of DNA with labeled consecutive nucleotides of a specific sequence and identify the labeled location in the DNA, forced linearization of the DNA random coil is performed. Some technologies to do this use nanoscale fluidic channels, through which DNA is forced to pass through, thereby being linearized. Nanochannels have a diameter of about 50 nm, several times larger than the diameter of double-stranded DNA, resulting in limits in linearly unfolding DNA, because at least two segments of the DNA may often overlap in a nanochannel region. Even with nanopores, it can be difficult to create a highly reproducible, sensitive DNA mapping environment unless the pore size is small enough to allow migration of just one segment of the DNA. Therefore, in genome research methodologies such as the above-described pore-based sequencing and optical DNA mapping, linearizing DNA chains is highly important, and there is a demand for the development of a system for stably linearizing DNA using the above-described advantages of nanoparticles.

### SUMMARY

Provided is a nanoparticle-nucleic acid complex.

Provided is a method of linearizing a target nucleic acid with the nanoparticle-nucleic acid complex.

Additional aspects will be set forth in part in the description which follows and, in part, will be apparent from the description, or may be learned by practice of the presented embodiments.

According to an aspect of the invention, a nanoparticle-nucleic acid complex including a nanoparticle bound with at least two protection sequence units and one probe sequence unit, wherein each of the at least two protection sequence units may include a single-stranded nucleic acid having an arbitrary base sequence, one terminus of which is bound to the nanoparticle; the probe sequence unit may include a single-stranded nucleic acid having a nucleotide sequence substantially complementarily binding to a first domain of a linking sequence unit, one terminus of the probe sequence unit being bound to the nanoparticle; and the linking sequence unit may include the first domain, which includes a single-stranded nucleic acid having a nucleotide sequence substantially complimentarily binding to the probe sequence unit, and a second domain including a single-stranded nucleic acid having a nucleotide sequence substantially complementarily binding to a terminus site of a target nucleic acid. The two protection sequence units need not be identical in sequence.

The nanoparticle-nucleic acid complex may include at least two protection sequence units and one probe sequence unit, which are bound to nanoparticles.

The probe sequence unit may include a single-stranded nucleic acid having a nucleotide sequence substantially complementarily binding to a first domain of a linking sequence unit, one terminus of the probe sequence unit being bound to the nanoparticle.

The linking sequence unit may include the first domain, which includes a single-stranded nucleic acid having a nucleotide sequence substantially complimentarily binding to the probe sequence unit, and a second domain that includes a single-stranded nucleic acid having a nucleotide sequence substantially complementarily binding to a terminus site of a target nucleic acid. The linking sequence unit may mediate binding of the nanoparticle-nucleic acid complex and the target nucleic acid.

### BRIEF DESCRIPTION OF THE DRAWINGS

These and/or other aspects will become apparent and more readily appreciated from the following description of the embodiments, taken in conjunction with the accompanying drawings of which:

FIG. 1 is a schematic diagram of a nanoparticle-nucleic acid complex according to an embodiment;

FIG. 2 is a schematic view illustrating a method of linearizing a target nucleic acid with the nanoparticle-nucleic acid complex, according to an embodiment; and

FIG. 3 is an electron microscopic image showing one-to-one binding of a 50-nm nanoparticle-nucleic acid complex to a 20-nm nanoparticle bound to a target nucleic acid.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Disclosed herein is a nanoparticle-nucleic acid complex including a nanoparticle bound with at least two protection sequence units and one probe sequence unit. In some embodiments, the nanoparticle-nucleic acid complex further includes a linking sequence unit having a first domain and a second domain, wherein the first domain is hybridized to the probe sequence unit.

As used herein, the term “nucleic acid” refers to a polymer of nucleotides and is equivalent to “polynucleotide”. The nucleic acid may include deoxyribonucleic acid (DNA, e.g., gDNA or cDNA) and/or ribonucleic acid (RNA), peptide nucleic acid (PNA), or locked nucleic acid (LNA). Nucleotides, which are the basic building blocks of nucleic acids, include deoxyribonucleotides and ribonucleotides. Nucleotides may be not only natural nucleotides but also nucleotide analogues including a modified sugar or base moiety. Natural deoxyribonucleotides include 4 types of bases: adenine (A), thymine (T), guanine (G), and cytosine (C). Ribonucleotides generally include a base that is C, A G, or uracil (U). A nucleic acid may be a single-stranded or double-stranded polymer.

The term “nanoparticle” refers to a particle having a diameter of about 1 nm to about 1000 nm. The nanoparticle may be a metal nanoparticle, a metal/metal core shell complex including a metal nanoparticle core and a metal shell surrounding the metal core, a metal/non-metal core shell complex including a metal nanoparticle core and a non-metal shell surrounding the metal core, or a non-metal/metal core shell complex including a non-metal nanoparticle core and a metal shell surrounding the non-metal core. In some embodiments, the metal may be selected from among, but not limited to, gold, silver, copper, aluminum, nickel, palladium, platinum, magnetic iron, and oxides thereof. The non-metal may be selected from among, but not limited to, silica, polystyrene, latex, and acrylate-based materials.

The probe sequence unit may be a single-stranded nucleic acid, one terminus of which may be bound to the nanoparticle. To bind to the nanoparticle, the probe sequence unit may further include, in addition to a single-stranded nucleic acid, a spacer via which the probe sequence unit may be bound to the nanoparticle. In some embodiments, the probe sequence unit includes a single-stranded nucleic acid and may further include a spacer having one terminus which may bind the probe sequence unit to a surface of the nanoparticle and another terminus that may be bound to the single-stranded nucleic acid. In some embodiments, the spacer may be selected from among, but is not limited to, a homopolynucleotide including a base selected from among A, G, C, T, and U; polyethylene glycol (PEG); and a combination thereof.

In some embodiments, the spacer may further include a functional group mediating binding to the nanoparticle. The functional group may be selected from among, but is not limited to, an amine group, a carboxylic group, a thiol group, a phosphoric acid group, and a combination thereof.

Either the 5′-terminus or the 3′-terminus of the single-stranded nucleic acid of the probe sequence unit may orient toward the nanoparticle surface upon binding of the probe sequence unit to the nanoparticle, depending on the sequence characteristics of the target nucleic acid to be linearized.

A linking sequence unit may include a first domain and a second domain.

The single-stranded nucleic acid of the probe sequence unit may include a nucleotide sequence substantially complementary to the first domain of a linking sequence unit. A complementary sequence to a single-stranded nucleic acid may include a perfectly complementary sequence or a substantially complementary sequence. The term “substantially complementary sequence” refers to a sequence hybridizable with the nucleotide sequence of the target nucleic acid under common hybridization stringency conditions in the art. The stringency conditions may be created by adjusting temperature, ionic strength (buffer concentration), or using a compound such as an organic solvent, and may depend on a sequence to hybridize. In some embodiments, the stringency condition may be a) washing at 50° C. with a solution of 0.015 M sodium chloride/0.0015 M sodium citrate/0.1% sodium dodecyl sulfate (SDS), and b) hybridization in a hybridization buffer (50% formamide, 2×SSC, and 10% dextran sulfate) at 55° C. and washing at 55° C. with an EDTA-containing 0.1×SSC.

The second domain of the linking sequence unit may include a single-stranded nucleic acid substantially complementary to a terminal site of the target nucleic acid.

The term “target nucleic acid” indicates a polymer of nucleotides to be analyzed. The target nucleic acid may be any double-stranded polynucleotide that has a single-stranded terminal site. Also, the target nucleic acid may be any single stranded polynucleotide. The target nucleic acid may have a length of about 10 bp to 1000 kb, and in some embodiments, may have a length of about 20 bp to about 500 kb. As used herein, the term “terminal site of a target nucleic acid” refers to an overhanging single-stranded nucleotide region of a few to tens of nucleotides at a 5′-terminus or a 3′-terminus of the double-stranded target nucleic acid. In some embodiments, the terminal site of the target nucleic acid may include a single-stranded polynucleotide region of about 2 nucleotides to about 50 nucleotides, and in some embodiments, of about 3 nucleotides to about 30 nucleotides. The single-stranded terminal site of a target nucleic acid may be inherently present on the target nucleic acid or can be made by any method known in the art.

The linking sequence unit may be obtained by modifying the nucleotide sequence of the second domain according to the nucleotide sequence of the terminal site of the target nucleic acid such that the second domain is substantially complementary to the nucleotide sequence of the terminal site of the target nucleic acid to permit hybridization between the second domain of the linking sequence unit and the terminal site of the target nucleic acid.

In some embodiments, a protection sequence unit may include a single-stranded nucleic acid having any nucleotide sequence, one terminus of which is or can be bound to a nanoparticle.

The single-stranded nucleic acid sequence of the protection sequence unit prevents the probe sequence unit from hybridizing to the terminal site of the target nucleic acid. In principle, the single-stranded nucleic acid of the protection sequence unit may have any nucleotide sequence. However, in some embodiments, the single-stranded nucleic acid of the protection sequence unit has a nucleotide sequence substantially complementary to the terminal site of the target nucleic acid to perform its protecting function.

In some embodiments, similar to the probe sequence unit, the protection sequence unit may further include a spacer at one terminus of the single-stranded nucleotide sequence, to permit binding of the protection sequence unit to the nanoparticle via the spacer. In some embodiments, the protection sequence unit includes a spacer, one terminus of which may bind the protection sequence unit to a surface of the nanoparticle and the other terminus of which may be bound to the single-stranded nucleic acid of the protection sequence unit. In some embodiments, the spacer may further include a functional group mediating binding to the nanoparticle. Detailed descriptions of the spacer and the functional group are provided above.

In some embodiments, the number of protection sequence units bound to the nanoparticle may vary but the number should be in the range of at least 2 to less than or equal to 3000. For example, for gold nanoparticles, the number of polynucleotides that are bound to the gold nanoparticles may vary depending on salt concentration, the type and length of the spacer, the size of the nanoparticles, and reaction conditions. The size of the nanoparticles is a primary factor relative to the others. For example, the number of nanoparticle-binding polynucleotides may be about 75 for gold nanoparticles having a diameter of about 15 nm, about 100 for those having a diameter of about 20 nm, about 200 for those having a diameter of about 100 nm, about 800 for those having a diameter of about 50 nm, and about 2300 for those having a diameter of about 80 nm. Therefore, by adjusting reaction conditions and a reaction equivalent of the nanoparticles and polynucleotides to be bound thereto according to the size of the nanoparticles to use, the nanoparticle-nucleic acid complex may be prepared.

According to another embodiment, a method of linearizing a target nucleic acid may include contacting a nanoparticle-nucleic acid complex and a target nucleic acid to hybridize a second domain of a linking sequence unit of the nanoparticle-nucleic acid complex to a terminal site of the target nucleic acid, wherein the target nucleic acid is substantially duplex with the proviso that the terminal site of the target nucleic acid is single-stranded; covalently binding the probe sequence unit and the linking sequence unit of the nanoparticle-nucleic acid complex to the target nucleic acid hybridized adjacent to the probe sequence unit and the linking sequence unit of the nanoparticle-nucleic acid complex to obtain a covalently bound structure; and applying a physical force to the bound structure to linearly unfold the target nucleic acid.

Each operation of the nucleic acid linearization method will be described in greater detail below.

The method may include contacting the nanoparticle-nucleic acid complex and the target nucleic acid to hybridize a second domain of a linking sequence unit of the nanoparticle-nucleic acid complex and a terminal site of the target nucleic acid;

The contacting is such as to permit hybridization of the single-stranded nucleic acid of the second domain of the linking sequence unit to the single-stranded terminal site of the target nucleic acid, and may be performed in vitro in an appropriate buffer solution under stringency conditions known in the art. Detailed descriptions of the target nucleic acid and the terminal site thereof are provided above. In some embodiments, the terminal site of the target nucleic acid may include a single-stranded polynucleotide of about 2 nucleotides to about 50 nucleotides, and in some other embodiments, of about 3 nucleotides to about 30 nucleotides. The target nucleic acid may be any substantially double-stranded polynucleotide that is long enough for linearization, for example, having a length of about 10 base pairs (bp) to about 1000 kb, and in some other embodiments, having a length of 20 bp to about 500 kb.

Then, the method may include covalently binding the probe sequence unit and the linking sequence unit of the nanoparticle-nucleic acid complex to the target nucleic acid which is hybridized adjacent to the probe sequence unit and the linking sequence unit of the nanoparticle-nucleic acid complex to obtain a bound structure. In some embodiments, the covalent binding is achieved by ligation of the end of one strand of the target nucleic acid adjacent to the end of the linking sequence unit and ligation of the end of the terminal site of the target nucleic acid adjacent to the end of the nucleic acid sequence of the probe sequence unit.

The term “hybridized adjacent to” refers to hybridization of the probe sequence unit and the terminal site of the target nucleic acid, respectively, to the first and second domains, respectively, of the linking sequence unit resulting in a “nick”, or discontinuity, in the sugar-phosphate backbone of the strand composed of the probe sequence unit and the terminal site of the target nucleic acid. When the probe sequence unit is hybridized to the linking sequence unit adjacent to the terminal site of the target nucleic acid, either one of the adjacent opposing termini of the probe sequence unit and the target nucleic acid may inherently have a terminal phosphate group permitting covalent linkage of the probe sequence unit and the target nucleic acid by creating a phosphodiester bond between the 3′ hydroxyl of one and the 5′ phosphate of the other in the presence of a ligase. Alternatively, if the 5′ terminus present at the discontinuity between the adjacent probe sequence unit and the target nucleic acid lacks a phosphate group, a phosphate may be introduced to the 5′ terminus to permit the ligase reaction. As one of ordinary skill in the art would realize, depending on the orientation of sequences in forming the nanoparticle-nucleic acid complex, the 5′-terminus at this nick could be on the probe sequence unit or on the terminal site of the target nucleic acid. This may also apply to covalent binding of the strand of the target nucleic acid aligned adjacent to the linking sequence unit.

The term “ligase” generically refers to a group of enzymes catalyzing the linkage of two molecules, often using ATP as the energy donor. DNA ligases close nicks, or discontinuities, in the sugar phosphate backbone of one strand of duplex DNA using ATP by creating a phosphodiester bond between adjacent 3′-hydroxyl and 5′-phosphate ends on the strand, with release of pyrophosphate and AMP. The ligase helps form a phosphodiester bond between the 5′-phosphate group and the 3′-hydroxyl group of two adjacent nucleotides to link the nucleotides. Thus, covalent binding of the nanoparticle-nucleic acid complex to the target nucleic acid may be achieved using a ligase, which facilitates binding of one strand of the double-stranded target nucleic acid to the second domain of the linking sequence unit as well as bonding of the opposite strand with the terminal site of the target nucleic acid to the probe sequence unit, thereby leading the nanoparticle-nucleic acid complex to form a covalently bound structure with the target nucleic acid.

In some embodiments, a moiety such as biotin may be attached to the terminus of the target nucleic acid which is not bound with the nanoparticle-nucleic acid complex, to facilitate binding to a surface of other nanoparticles or other particles.

Finally, the method may include applying a physical force to the bound structure to linearly unfold the target nucleic acid.

The target nucleic acid may be linearly unfolded by applying a physical force to the nanoparticle. In some embodiments, the physical force may be selected from among, but is not limited to, a magnetic force, an electric force, a centrifugal force, gravity, and a fluidic force. In another embodiment, the linear unfolding may be performed within a nanochannel. After the linear unfolding of the target nucleic acid, analyzing the nucleotide sequence of the target nucleic acid or mapping the target nucleic acid may be performed depending on the experimental purpose.

According to another embodiment, a kit for linearizing a target nucleic acid includes the nanoparticle-nucleic acid complex.

The kit might include the nanoparticle-nucleic acid complex. Since already described above, the nanoparticle-nucleic acid complex and the method of linearizing a target nucleic acid by using the nanoparticle-nucleic acid complex may not be described here again, for convenience of description.

The present invention will be described in further detail with reference to the following figures. These figures are for illustrative purposes only and are not intended to limit the scope of the invention.

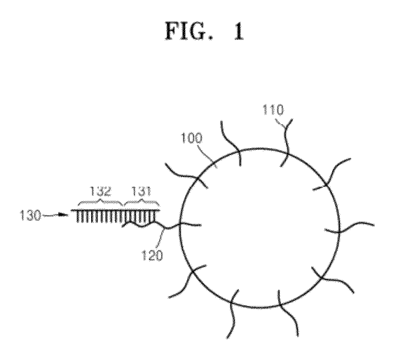

FIG. 1 illustrates a nanoparticle-nucleic acid complex according to an embodiment of the invention. FIG. 1 shows a nanoparticle-nucleic acid complex in which protection sequence unit 110 and probe sequence unit 120 are each bound to nanoparticle 100. Additionally, FIG. 1 shows the nanoparticle-nucleic acid complex also includes linking sequence unit 130, comprising first domain 131 and second domain 132, with first domain 131 hybridized to probe sequence unit 120.

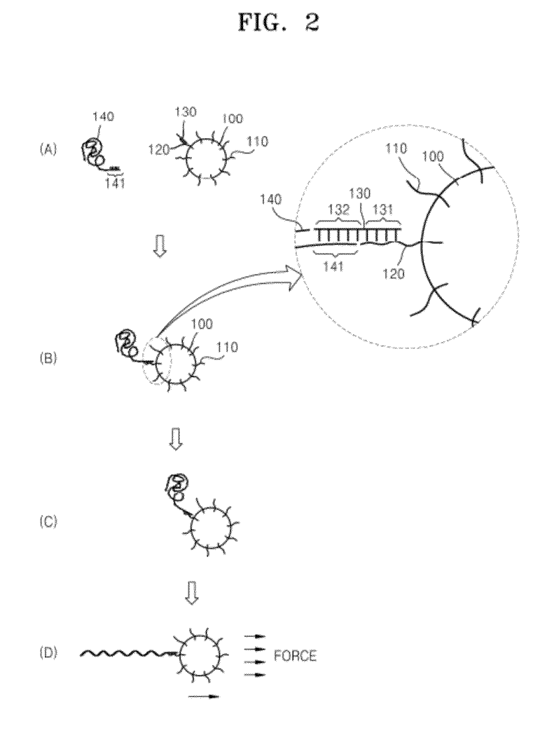

FIG. 2 is a schematic view illustrating a method of linearizing a target nucleic acid 140 with the nanoparticle-nucleic acid complex of FIG. 1, according to an embodiment of the invention.

Referring to FIG. 2, in panel A, the target nucleic acid 140 is brought into contact with the nanoparticle-nucleic acid complex of FIG. 1 in a buffer solution. Target nucleic acid 140 includes a single-stranded terminal site 141 substantially complementary to second domain 132 of linking sequence unit 130 of the nanoparticle-nucleic acid complex, therefore terminal site 141 of the target nucleic acid 140 may be hybridized with second domain 132 of the nanoparticle-nucleic acid complex, as shown in panel B. In the enlargement of panel B, target nucleic acid is shown to be double-stranded except for terminal site 141. A protection sequence unit 110 of the nanoparticle-nucleic acid complex may be a polynucleotide having any nucleotide sequence which is unable to hybridize to terminal site 141 of target nucleic acid 140. As a result of the hybridization of probe sequence unit 120 with first domain 131 of linking sequence unit 130 and hybridization of target nucleic acid terminal site 141 with second domain 132 of linking sequence unit 130, terminal site 141 of target nucleic acid 140 and probe sequence unit 120 are positioned adjacent to each other as one nicked strand of a duplex structure including linking sequence unit 130 as the opposite strand (FIG. 2 (B)). Then, with addition of a ligase, the nicked strand composed of the hybridized probe sequence unit 120 and terminal site 141 of the target nucleic acid are ligated together. Similarly, the strand in the hybrid duplex including the linking sequence unit 130 and the end of the duplex region of target nucleic acid 140 can be ligated together with addition of a ligase to form a double-stranded nucleic acid with no nicks. Afterwards, a controlled physical force such as an electric force, a magnetic force, and the like is applied to attract the nanoparticle 100, thereby allowing the attached target nucleic acid 140 to be linear unfolded as shown in panel (D). Application of the controlled physical force may be conducted in a device such as a nanochannel.

The following examples are for illustrating a method of synthesizing a nanoparticle-nucleic acid complex, and showing results of one-to-one binding a target nucleic acid to the nanoparticle-nucleic acid complex.

### Example 1

### Preparation of Polynucleotides to be Bound to Nanoparticle Surface

A 5′-modified polynucleotide 5′-HO-(CH₂)₃-S-S-A₁₀-PEG18-TGATGAATTCTACTG-3′ (SEQ ID No. 1, available from Integrated DNA Technologies, Inc., USA) was added to 0.1 M dithiothreitol, and then left at room temperature for 2 hours to obtain the deprotected 5′-thiol-modified oligonucleotide. The deprotection solution was subjected to chromatography through a NAP-5 column (Sephadex G-25 medium, DNA grade) to purify the deprotected polynucleotide 5′-HS-A₁₀-PEG18-TGATGAATTCTACTG-3′ (SEQ ID No. 1) for use as the probe sequence unit.

A second polynucleotide, 5′-HS-A₁₀-PEG18-ACTCATTAAGATTAC-3′ (SEQ ID No. 2), for use as a protection sequence unit was prepared from a 5′-modified polynucleotide (5′-HO-(CH₂)₃-S-S-A₁₀-PEG18-ACTCATTAAGATTAC-3′, SEQ ID No. 2) in the same manner as described above. Afterwards, the polynucleotides in solution were quantized by measuring absorbance using a UV-Visible spectrometer.

In these polynucleotides, PEG stands for polyethylene glycol (PEG), and A10 is a 10-mer of adenosine monophosphate.

### Example 2

### Synthesis of Nanoparticle-Nucleic Acid Complex

A total of 4 nmol of the two reduced polynucleotides prepared in Example 1 (at a ratio of the protection sequence unit to the probe sequence unit=799:1) was added to 1 ml gold nanoparticles (0.04 nM) having a 50 nm diameter (available from Ted Pella, Inc., U.S.A.), and then stirred in an orbital shaker for 20 minutes. After mixing, the concentration of the solution of metal nanoparticles and polynucleotides was adjusted with a 100 mM phosphate buffer solution and a 10% sodium dodecyl sulphate (SDS) solution to contain 10 mM phosphate buffer and 0.01% SDS, and then stirred in an orbital shaker for 20 minutes. A quantity of 2M NaCl solution was added to the solution in four aliquots at 20-minute intervals until a final salt concentration of the mixed solution reached 0.3M NaCl. Then, the solution was slowly stirred for 12 hours, centrifuged twice (at 4000 rpm for 13 minutes and at 8000 rpm for 15 minutes), and washed with 1 ml of a 10 mM phosphate buffer solution twice. After further centrifugation (at 4000 rpm for 15 minutes), the precipitate from the centrifugation was re-suspended in 0.3 M phosphate buffered saline (PBS). Absorbance of the resulting suspension was measured using a UV spectrometer to determine that the concentration of the nanoparticle-nucleic acid complex was about 0.04 nM.

### Example 3

### Determination of Binding Ratio of Nanoparticle-Nucleic Acid Complex to Target Nucleic Acid

The binding ratio of the nanoparticle-nucleic acid complex prepared in Example 2 to a target nucleic acid was determined.

A polynucleotide having nucleotide sequence 5′-GGGCGGCGACCTCATTAGAATTCATCA-3′ (SEQ ID No. 3) was used as f a linking sequence unit. A polynucleotide having nucleotide sequence 5′-AGGTCGCCGCCC-3′ (SEQ ID No. 4), complementary to the 5′ 50% of the linking sequence unit, was used as the target nucleic acid.

The target nucleic acid was bound to 20-nm gold nanoparticles together with a polynucleotide having an arbitrary sequence in a 99:1 ratio using methods generally in accordance with those described in Example 2, thereby preparing target nucleic acid-bound nanoparticles, which are smaller than the nanoparticle-nucleic acid complex of Example 2, including 50-nm gold nanoparticles. The difference in size of the two different nanoparticle complexes permit the binding pattern of the target nucleic acid on the 20 nm nanoparticles to the 50 nm nanoparticle-nucleic acid complex to be identified visually using a microscope.

The target nucleic acid-bound nanoparticles and the linking sequence unit in a 1:1 ratio, were added to 100 μl of the 0.04 nM 50 nm nanoparticle-nucleic acid complex prepared in Example 2 at a 5,000 times higher concentration. The mixture was put in a 65° C. water bath for 5 minutes, and then slowly stirred at room temperature for 2 days. Subsequently, the reaction product was centrifuged at 4000 rpm for 15 minutes, and unhybridized linking sequence unit and target nucleic acid-bound nanoparticles were removed from the precipitated hybridization complex. T4 DNA ligase in buffer solution (0.5M Tris-HCl, 0.1M MgCl2, 10 mM ATP, pH 7.5) was added to the hybridized reaction product (2 pmol) in a 1:9 volumetric ratio, and permitted to react with stirring at 16° C. for 30 minutes.

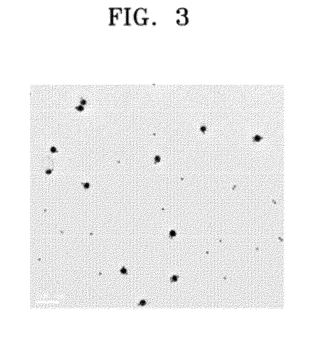

The hybridized reaction product was visualized using an electron microscope. FIG. 3 is an electron microscope image that shows that target nucleic acid-bound nanoparticles were bound one-to-one to the 50 nm nanoparticle-nucleic acid complex. In particular, about 60% or more of the 50 nm nanoparticle-nucleic acid complex were bound one-to-one with the 20 nm target nucleic acid bound nanoparticles.

As described above, according to one or more of the above embodiments of the present disclosure, by using a nanoparticle-nucleic acid complex, and a method of linearizing a target nucleic acid with the nanoparticle-nucleic acid complex, nucleotide sequence analysis and mapping of a target nucleic acid may be efficiently performed.

The terminology used herein is for the purpose of describing particular embodiments only and is not intended to be limiting of the invention. The terms “a” and “an” do not denote a limitation of quantity, but rather denote the presence of at least one of the referenced item. The terms “comprising”, “having”, “including”, and “containing” are to be construed as open-ended terms (i.e. meaning “including, but not limited to”). The modifier “about” used in connection with a quantity is inclusive of the stated value and has the meaning dictated by the context (e.g., includes the degree of error associated with measurement of the particular quantity). In some embodiments, the degree of error or variation in the particular quantity denoted by “about” is ±10% of the particular quantity, specifically ±5% of the particular quantity, more specifically ±1% of the particular quantity.

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A nanoparticle-nucleic acid complex comprising
a nanoparticle bound to a plurality of protection sequence units and one probe sequence unit,
wherein a protection sequence unit comprises a single-stranded nucleic acid having an arbitrary base sequence, one terminus of which is bound to the nanoparticle and
the probe sequence unit comprises a single-stranded nucleic acid having a nucleotide sequence substantially complementary to a first domain of a linking sequence unit, one terminus of the probe sequence unit being bound to the nanoparticle; and
the linking sequence unit, wherein the linking sequence unit comprises
the first domain, hybridized to the probe sequence unit, and
a second domain comprising a single-stranded nucleic acid having a nucleotide sequence substantially complementary to a terminal site of a target nucleic acid.

2. The nanoparticle-nucleic acid complex of claim 1, wherein the protection sequence unit further comprises a spacer, one terminus of which is bound to a surface of the nanoparticle and the other terminus of which is bound to the single-stranded nucleic acid of the protection sequence unit.

3. The nanoparticle-nucleic acid complex of claim 1, wherein the probe sequence unit further comprises a spacer, one terminus of which is bound to a surface of the nanoparticle and the other terminus of which is bound to the single-stranded nucleic acid of the probe sequence unit.

4. The nanoparticle-nucleic acid complex of claim 2, wherein the spacer is selected from the group consisting of a homopolynucleotide; polyethyleneglycol (PEG); and a combination thereof.

5. The nanoparticle-nucleic acid complex of claim 3, wherein the spacer is selected from the group consisting of a homopolynucleotide; polyethyleneglycol (PEG); and a combination thereof.

6. The nanoparticle-nucleic acid complex of claim 2, wherein the spacer comprises a functional group mediating the binding to the nanoparticle.

7. The nanoparticle-nucleic acid complex of claim 3, wherein the spacer comprises a functional group mediating the binding to the nanoparticle.

8. The nanoparticle-nucleic acid complex of claim 6, wherein the functional group is selected from the group consisting of an amine group, a carboxylic group, a thiol group, a phosphoric acid group, and a combination thereof.

9. The nanoparticle-nucleic acid complex of claim 7, wherein the functional group is selected from the group consisting of an amine group, a carboxylic group, a thiol group, a phosphoric acid group, and a combination thereof.

10. The nanoparticle-nucleic acid complex of claim 1, wherein the nanoparticle has a diameter of about 1 nm to about 1000 nm.

11. The nanoparticle-nucleic acid complex of claim 1, wherein the nanoparticle comprises a metal nanoparticle, a metal/metal core shell complex including a metal nanoparticle core and a metal shell surrounding the metal nanoparticle core, a metal/non-metal core shell complex including a metal nanoparticle core and a non-metal shell surrounding the metal nanoparticle core, or a non-metal/metal core shell complex including a non-metal nanoparticle core and a metal shell surrounding the non-metal nanoparticle core.

12. The nanoparticle-nucleic acid complex of claim 11, wherein the metal is selected from the group consisting of gold (Au), silver (Ag), copper (Cu), aluminum (Al), nickel (Ni), palladium (Pd), platinum (Pt), magnetic iron, and an oxide thereof.

13. The nanoparticle-nucleic acid complex of claim 11, wherein the non-metal is selected from the group consisting of silica, polystyrene, latex, and an acrylate-based material.

14. The nanoparticle-nucleic acid complex of claim 1, wherein the plurality of protection sequence units consists of a number of protection sequence units greater than or equal to 2 or less than or equal to about 3000.

15. A method of linearizing a target nucleic acid, the method comprising:
contacting the nanoparticle-nucleic acid complex of claim 1 with the target nucleic acid such that the second domain of the linking sequence unit hybridizes to the terminal site of the target nucleic acid, wherein the target nucleic acid is a duplex except for the single-stranded overhanging terminal site;
ligating the probe sequence unit to the terminal site of the target nucleic acid hybridized to the linking sequence unit immediately adjacent to the probe sequence unit and ligating the linking sequence unit to the strand of the target nucleic acid opposite to the strand of the terminal site to form covalently bound target-nanoparticle structure; and
applying a physical force to the target-nanoparticle structure to linearly unfold the target nucleic acid.

16. The method of claim 15, wherein the terminal site of the target nucleic acid comprises a single-stranded polynucleotide of a length from about 3 nucleotides to about 30 nucleotides.

17. The method of claim 15, wherein the target nucleic acid has a length of about 20 bp to about 500 kb.

18. The method of claim 15, wherein ligating is performed using a ligase.

19. The method of claim 15, wherein applying a force to the target-nanoparticle structure is performed with the target-nanoparticle structure in a nanochannel.

20. The method of claim 15, wherein the physical force is selected from the group consisting of a magnetic force, an electric force, a centrifugal force, gravity, and a fluidic force.

21. A kit for linearizing a target nucleic acid, comprising the nanoparticle-nucleic acid complex of claim 1.
